(19) United States
(12) Patent Application Publication
(10) Pub. No.: US 2006/0039966 A1
(43) Pub. Date: Feb. 23, 2006

(54) Dosage delivery apparatus for improving user acceptance of oral supplements and medicaments and methods for manufacturing same

(76) Inventors: Bruce A. Miller JR., Mt. Bethel, PA (US); Olav E. Sandnes, Mt. Bethel, PA (US)

Correspondence Address: PATE PIERCE & BAIRD, 215 SOUTH STATE STREET, SUITE 550, PARKSIDE TOWER, SALT LAKE CITY, UT, 84111, US

(21) Appl. No.: 11/201,764

(22) Filed: Aug. 11, 2005

Related U.S. Application Data
(60) Provisional application No. 60/600,975, filed on Aug. 12, 2004.

(51) Int. Cl. A61K 35/60; A61K 9/48; A61K 9/20

(52) U.S. Cl. 424/451; 424/464; 424/523

(57) ABSTRACT

Disclosed herein are methods for improving user acceptance of an oral dosage form containing an active ingredient for use as a supplement and/or medicament by masking undesirous flavors, odors, tastes, smells, and/or palatability associated with certain active ingredients. A user may be a human and/or animal in need of a particular active ingredient. A preferred embodiment of the methods of the present invention may include: (1) selecting the oral dosage form; (2) incorporating an active ingredient into the oral dosage form; and (3) applying at least one substance to improve user acceptance of the oral dosage form. A substance to improve user acceptance of the oral dosage form may include a fragrance, flavorant, and/or appearance modification agent. Application of a substance to improve user acceptance and the organoleptic qualities of the oral dosage form may be accomplished by introducing the oral dosage form into a polishing and/or tumbler apparatus, washing the dosage form, and/or applying a coating to the oral dosage form. A preferred embodiment of the present invention improves user acceptance and the organoleptic qualities of the oral dosage form by suppressing unpleasant odors, flavors, taste, and/or palatability while not altering the physical parameters of the oral dosage form and being substantially more economical to produce. A preferred embodiment of the present invention may result in a substance to improve user acceptance being applied to the outer and/or inner surface of an oral dosage form.

### RELATED APPLICATIONS

This application claims the benefit of U.S. Provisional Patent Application Ser. No. 60/600,975, filed Aug. 12, 2004, and entitled “DOSAGE DELIVERY APPARATUS FOR IMPROVING USER ACCEPTANCE OF ORAL SUPPLEMENTS AND MEDICAMENTS AND METHODS FOR MANUFACTURING SAME,” which is incorporated herein by reference.

### BACKGROUND

1. Field of the Invention

This invention relates to methods to improve user acceptance of an oral dosage form containing an active ingredient for use as a supplement and/or medicament and more particularly, to novel dosage delivery apparatus for improving user acceptance of an oral dosage form of supplements and medicaments and methods for manufacturing the same, which facilitate the masking of undesirous flavors, odors, tastes, smell, and/or palatability (characterized herein as “organoleptic qualities”) associated with certain active ingredients.

2. The Background Art

As appreciated by those skilled in the art, certain active ingredients that may be included in nutritional supplements and/or medicaments may have any number of physical and/or chemical characteristics that may make the active ingredient undesirable for consumption by a user. In many circumstances, a particular active ingredient may be known to have significant health benefits, yet the demonstrated health benefits may not be persuasive enough to overcome the reluctance on the part of a user to consume the active ingredient. Reluctance to consume by a user may be the result of a noxious odor, taste, flavor, palatability, and/or unpleasant smell of the ingredient, supplement, and/or medicament.

In some conditions, reluctance to consume a dosage formulation results in decreased compliance with a particular health supplement and/or therapeutic regimen. This may have a profound impact on the health and/or well-being of a targeted user or patient. In addition, there are also regional, cultural, or societal preferences that may influence user acceptance of an oral dosage form containing a supplement and/or medicament. These differences may often result in additional production costs to produce dosage formulations with the same active ingredient, but with different formulation parameters as required by regional, cultural, or societal preferences.

Pharmaceutical science is abroad discipline, however, one aspect of this art involves developing new dosage forms and ways to deliver active ingredients to a user. As appreciated, those skilled in the pharmaceutical arts have long recognized the impact user preference may have on consumption and/or ingestion of a dosage formulation containing particular active ingredients. For example, in the mid-1800's, pharmacists and pharmaceutical scientists in France began to develop capsular dosage forms to aid patients in swallowing the active ingredient and also in an attempt to minimize unpleasant tastes and/or odors associated with certain active ingredients.

Active ingredients based on oils and other materials containing hydrocarbon chains are generally inclined to produce putrid or otherwise disagreeable odors and/or tastes. Pharmaceutical technologies directed to placing the active ingredients into capsules and/or microencapsulation processes have had only limited success in reducing these undesirous odors and/or tastes of certain active ingredients. As appreciated by those skilled in the medical arts, an oral dosage form containing an oil based active ingredient may often be chilled prior to administration to a patient. One reason for this action may be to reduce the volatility of oil and reduce the amount of vapors an oral dosage form may emanate.

Coating mechanisms are also well-known in the art as providing a means to impart additional taste and/or dissolution characteristics to oral dosage forms. Coating technology has primarily involved the application of sugar-coatings, timed-release coatings, and enteric-protective coatings. More particularly, coating technology has taught the means to alter the dissolution of the oral dosage form and release of the active ingredients. Prior art coating technologies of the prior art, however, have not taught or disclosed the use of a coating for improving the organoleptic qualities of oral dosage forms (e.g., masking taste, odor, flavor, and/or smell, whereby improving palatability).

In spite of profound advances in overall pharmaceutical science technology, there has been relatively minimal advancement in capsular technologies. More particularly, there have been few advances in further reducing undesirable odor, taste, smell, or flavor associated with certain active ingredients. Although soft elastic capsule (SEC) technology has found increased use as an oral dosage form, it may not have improved ability over hard gelatin capsules to mask unpleasant odor and taste. In addition, SEC may not facilitate any real significant ability to improve the organoleptic qualities of the active ingredients.

In an attempt to address the noxious odor of some ingredients, those skilled in the art have disclosed the direct inclusion and/or incorporation of pharmaceutical excipients in an attempt to mask odor, flavor, smell, or taste into the composition materials which are used to manufacture the oral dosage form. These teachings, however, have largely focused on the alteration of the physical and/or chemical parameters of the oral dosage form by augmenting the dosage form composition itself by some means. These prior art efforts appear to add significant cost to the step of production and manufacturing of capsules, while not dramatically improving user preference. In addition, the apparatus and methods of the prior art tend to impart characteristics to the dosage delivery form that may alter the desirable pharmacokinetic parameters for dissolution, release, absorption, and/or bioavailability of the active ingredient into the circulation of a user.

Consistent with the foregoing state of the art and the disadvantages associated therewith, what is needed are dosage delivery apparatus for improving user acceptance of an oral dosage form containing an active ingredient for use as a supplement and/or medicament and methods for manufacturing the same. In addition, dosage delivery apparatus and methods for improving user acceptance of an oral dosage form are needed which are economical, have little or no impact on other desirable pharmacokinetic variables of the dosage form, and improve compliance in a user leading to improved health and/or well-being. Novel apparatus and methods providing useful, economical, efficacious, and safer alternatives to the prior art are needed. Such novel dosage delivery apparatus and methods are disclosed and taught herein.

### BRIEF SUMMARY AND OBJECTS OF THE INVENTION

A primary object of the present invention is to provide novel dosage delivery apparatus for improving user acceptance of an oral dosage form containing an active ingredient for use as a supplement and/or medicament and methods for manufacturing the same.

It is also an object of the present invention to provide novel dosage delivery apparatus for improving user acceptance of an oral dosage form containing an active ingredient for use as a supplement and/or medicament and methods for manufacturing the same in which a substance to improve user acceptance is applied to at least a portion of a surface of an oral dosage form.

It is a further object of the present invention to provide novel dosage delivery apparatus for improving user acceptance of an oral dosage form containing an active ingredient and method for manufacturing the same, wherein a fragrance, flavorant, or coloring agent is applied to a surface of an oral dosage form.

It is a still further object of the invention to provide novel dosage delivery apparatus for improving user acceptance of an oral dosage form containing an active ingredient and methods for manufacturing the same, which is economical to produce and manufacture.

In addition, it is an object of the present invention to provide novel dosage delivery apparatus for improving user acceptance of an oral dosage form containing an active ingredient and methods for manufacturing the same, in which the basic pharmacokinetic characteristics of the oral dosage form are substantially unaltered.

Moreover, it is an object of the present invention to provide novel dosage delivery apparatus for improving user acceptance of an oral dosage form containing an active ingredient and methods for manufacturing the same, wherein a substance to improve user acceptance is applied to an oral dosage form by polishing cloths in a tumbler apparatus.

Additionally, it is an object of the present invention to provide novel dosage delivery apparatus for improving user acceptance of an oral dosage form containing an active ingredient and methods for manufacturing the same, in which a substance to improve user acceptance is applied to an oral dosage form utilizing a spray coating process.

It is also an object of the present invention to provide novel methods for improving user acceptance of an oral dosage form containing an active ingredient selected from a nutritional supplement, natural product, pharmaceutical, food supplement, diagnostic agent, vitamin, mineral, botanical, herbal, marine product, and combinations thereof and methods for manufacturing the same.

Moreover, it is an object of the present invention to provide novel dosage delivery apparatus for improving user acceptance of an oral dosage form containing an active ingredient and methods for manufacturing the same, which include a substance to facilitate user acceptance that is applied to the outer surface and/or the inner surface of an oral dosage form to improve the organoleptic qualities or characteristics (e.g., flavor, smell, taste, and/or palatability) of the oral dosage form.

It is also an object of the present invention to provide novel dosage delivery apparatus for improving user acceptance of an oral dosage form containing an active ingredient for use as a supplement and/or medicament and methods for manufacturing the same, wherein a substance to facilitate user acceptance is applied by washing the oral dosage form with the substance and drying the oral dosage form to improve the organoleptic qualities or characteristics (e.g., flavor, smell, taste, and/or palatability) of the oral dosage form.

It is a further object of the present invention to provide novel dosage delivery apparatus for improving user acceptance of an oral dosage form containing an active ingredient and methods for manufacturing the same, wherein a substance to facilitate user acceptance is applied to a surface of an oral dosage form to improve the organoleptic qualities or characteristics (e.g., flavor, smell, taste, and/or palatability) of the oral dosage form whereby those consumers who have a hard time swallowing certain oral dosage forms and desire to chew the dosage form may do so, if desired.

Also, it is an object of the present invention to provide novel dosage delivery apparatus for improving user acceptance of an oral dosage form containing an active ingredient for use as a supplement and/or medicament and methods for manufacturing the same, wherein a user is a human and/or animal.

Consistent with the foregoing objects, and in accordance with the invention as embodied and broadly described herein, it has been found that novel methods for applying a substance to improve the organoleptic qualities (e.g., flavor, smell, taste, and/or palatability) of an oral dosage form are effective in improving user consumption. As appreciated, novel dosage delivery apparatus for improving user consumption of an oral dosage form containing an active ingredient and methods for manufacturing the same as taught by the present invention have not heretofore been taught or disclosed in the prior art. Accordingly, the present invention contemplates improving user compliance with an oral dosage form containing an active ingredient that has generally undesirous odor, taste and/or organoleptic qualities characteristics. Moreover, the present invention teaches a novel dosage delivery apparatus comprising a substance for improving user consumption of an oral dosage form that is not integrated into the oral dosage form, that does not substantially alter the dissolution characteristics or the release of the active ingredient into the circulation system of a user.

### DETAILED DESCRIPTIONS OF THE PREFERRED EMBODIMENTS

It will be readily understood that the apparatus and methods of the present invention, as generally described herein, could be arranged and designed in a wide variety of different configurations. Those of ordinary skill in the art will, of course, appreciate that various modifications to the details herein may be made without departing from the essential characteristics of the invention, as described. Thus, the following more detailed description of the embodiments of the apparatus and methods of the present invention is not intended to limit the scope of the invention, as claimed, but it is merely representative of the presently preferred embodiments of the invention.

Novel dosage delivery apparatus of the present invention may include oral dosages formulated as solid, liquid, powder, tablet, capsule, gel, soft gel, soft elastic capsule, granule, microgranule, emulsion, suspension, troche, tablet triturate, lozenge, and pill, or other means suitable for oral ingestion. In addition, novel dosage delivery apparatus of the present invention may include at least one active ingredient that is known to have a physical and/or chemical property that would make it undesirable for formulation into an oral dosage form. Such undesirable properties may include noxious odor, noxious taste, and/or unpleasant organoleptic qualities.

Unless otherwise defined, the technical, scientific, and medical terminology used herein has the same meaning as understood by those informed of the art to which this invention belongs. However, for the purposes of establishing support for various terms that are used in the present application, the following technical comments, definitions, and review are provided for reference.

“Compliance” may refer to the ability of a user to conform with instructions and/or guidelines established by another authority. In the medical or nutritional arts, compliance may refer to the ability of a user to conform with the recommendations of a physician, pharmacist, and/or other clinical authority in consuming a nutrient, nutritional supplement, and/or medicament.

“Organoleptic qualities” may refer to the sensory characteristics including, by way of example, odor, flavor, smell, taste, and/or palatability.

“Flavor” may refer to any of a group of sensory perceptions that may include smell, taste, texture, and/or appearance. Often, flavor may be used synonymously with stimulation of taste receptors on the tongue.

“Palatable” may refer to something agreeable to a user for purposes of consumption into the body. Often used interchangeably with items that are pleasant to the taste and implying desirability of a user to consume the item.

“Taste” may refer to a sensory perception involving excitation of gustatory receptors on the tongue designed to detect bitter, salty, sweet and/or sour sensations. In some prior art, it has been found that those skilled in the art sometimes use “taste” interchangeably with “flavor,” which are largely given the same meaning and interpretation.

“Smell” may be used interchangeably with “odor” and may refer to a sensory perception involving excitation of the olfactory nerves and, more particularly, stimulation of the sensory nerves of the nasal passages. Additional terms sometimes used to describe smell include fragrance, scent, and/or aroma.

As appreciated by those skilled in the art, dosage formulations may classified in many ways. Oral dosage forms may be the most widely utilized and available dosage formulation. Typically, these formulations are introduced into the body of a human and/or animal through the gastrointestinal tract (i.e., ingestion). Because of the consistency and general environment of the gastro-intestinal tract, it is not required that oral formulations be completely sterile products (i.e., no contamination by microorganisms and/or foreign particles). It is generally known to those skilled in the art that capsular oral dosage forms may provide some advantages in terms of ease in swallowing and user preference. However, not all the problems associated with administration of active ingredients are solved with capsular oral dosage forms. For instance, and not by limitation, capsules may not provide sufficient odor masking capability for some oily substances and may not be suitable for certain highly ionizable compounds which may result in localized tissue irritation.

Soft elastic capsules (SEC) also known as soft gelatin capsules and/or soft gels have recently received more attention for the encapsulation of liquid and/or oil formulations. Active ingredients in liquid and/or oil-based physical states may have an improved likelihood to emit an odor. Such an odor and/or taste may inhibit the user from introducing the oral dosage form into their mouth for ingestion. As discussed above, this may have substantial effects on health and/or well-being of the user if there is reluctancy to take the dosage form because of noxious odors and/or taste.

One presently preferred method of the present invention for manufacturing a capsular oral dosage form containing an active ingredient may include the steps of: (1) preparing the active ingredient; (2) introducing the active ingredient into a capsule; (3) finishing the capsule (i.e., sealing, dusting, polishing, washing, coating, sorting, banding, marking, and the like); and (4) packing the capsule. As appreciated, it may be desirous to impart additional characteristics to oral dosage formulations to improve user consumption. This may be particularly important with certain active ingredients in supplements and/or medicaments that are known or otherwise highly suspected to have important effects on health, quality-of-life, and/or well-being of a user yet have physical and/or chemical characteristics which reduce acceptance and consumption on the part of a targeted user.

Previous attempts by those skilled in the art to minimize the impact of these undesirous physical and/or chemical characteristics have been largely unsuccessful in mitigating the undesirous physical and/or chemical characteristic (i.e., unpleasant odor, taste, smell, flavor, or palatability). Similarly, prior art attempts generally introduce additional production costs into manufacturing a viable end product and may require integration into the formulation itself and therefore introduce additional undesirable characteristics to the oral dosage form (e.g., may alter dissolution of the dosage form, or may alter absorption of the active ingredient into the circulation of a user).

Marine products (i.e., compositions derived, isolated, and/or extracted from marine animals or plants) are widely believed to contain compounds which may have profound effects on the health, quality-of-life, and/or well-being of human and animal users. For example, and not by way of limitation, certain ocean fish are known to contain high quantities of polyunsaturated fatty acids (PUFA's), and more particularly omega-3 PUFA's.

Over the past few decades, PUFA's have been subjected to several clinical trials and results suggest these compounds have substantial utility in ameliorating and/or mitigating numerous health conditions, including, for example and not by limitation, cardiovascular disorders, neurological disorders, thromboembolic disorders, inflammatory and immune system disorders, joint and musculoskeletal disorders, and the like. Clinical and scientific investigations have revealed that humans and many animals have little or no capability to produce certain PUFA's through biochemical pathways. Therefore any potential health benefits from PUFA's in humans and many animals would require a human and/or animal to acquire the PUFA through dietary intake or administration of a supplement and/or medicament.

Recognizing the potential health benefits of PUFA's, those skilled in the art have sought out and commercialized exogenous sources of PUFA for supplementation in humans and animals. As previously mentioned, marine fish generally have high quantities of PUFA. An important challenge in supplementation of PUFA's by use of marine products is that many fish have particularly strong odors.

As appreciated by those skilled in the art, PUFA's may be composed of hydrocarbon chains of varying lengths and varying levels of unsaturated chemical bonds. PUFA's typically generate an odor, which may often function as a deterrent to consumption of marine products by a user. In particular, many human users have difficulty overcoming this undesirable property, (i.e., odor and taste) in spite of knowing the potential health benefit that may result from PUFA supplementation.

While PUFA's are an important example of the utility of the present invention, there are many other active ingredients for use in supplements and/or medicaments that may have undesirable physical and/or chemical properties. All such active ingredients are contemplated as falling within the spirit and scope of the present invention.

As disclosed and taught herein, the present invention discloses dosage delivery apparatus for improving consumer acceptance of an oral dosage form containing an active ingredient for use as a supplement and/or medicament and methods of manufacturing the same, wherein at least one of the active ingredients has at least one undesirable organoleptic quality (e.g., flavor, smell, taste, and/or palatability). In one presently preferred embodiment of the present invention, a method for improving user acceptance of an oral dosage form, may include the steps of: (1) selecting the oral dosage form; (2) incorporating an active ingredient into the oral dosage form; and (3) applying at least one substance to at least a portion of the outer surface of the oral dosage form to improve user acceptance of the oral dosage form. Improving user acceptance may include improving compliance of a user with a supplementation and/or therapeutic regimen. Moreover, a substance for improving user acceptance and the organoleptic qualities (e.g., flavor, smell, taste, and/or palatability) of an oral dosage form may include a fragrance, flavorant, and/or appearance modification agent. Similarly, the substance for improving user acceptance may be apply to an outer and/or inner surface of an oral dosage form.

An oral dosage form may be selected from the group consisting of a solid, liquid, powder, tablet, capsule, gel, soft gel, soft elastic capsule, granule, microgranule, emulsion, suspension, troche, tablet triturate, lozenge, and pill. In addition, an active ingredient may be selected from the group consisting of a nutritional supplement, natural product, pharmaceutical, food supplement, diagnostic agent, vitamin, mineral, botanical, herbal, and marine products.

In another presently preferred embodiment of the present invention a method for improving user acceptance of an oral dosage form may include the steps of: (1) selecting an active ingredient for delivery to a user; (2) selecting the oral dosage form; (3) incorporating the active ingredient into the oral dosage form; and (4) polishing the oral dosage form in a tumbler apparatus, wherein one or more polishing cloths are coated with at least one substance to improve user acceptance, and wherein the substance is transferred to at least a portion of the outer surface of the oral dosage form. For example, a fragrance may be applied to the polishing cloth(s) in a tumbler. Finished oral dosage forms may be introduced into a tumbler and for a specified duration of time may be tumbled in contact with one or more polishing cloths. This action may impart a pleasant fragrance (or otherwise neutralize an unpleasant fragrance) to a surface of an oral dosage form. A flavorant or other organoleptic (i.e. sensory) modifying agent may be substituted for a fragrance or used in combination with a fragrance on the polishing cloths.

In still another presently preferred embodiment of the present invention a method for improving user acceptance of an oral dosage form may include: (1) selecting an active ingredient for delivery to a user; (2) selecting the oral dosage form; (3) incorporating the active ingredient into the oral dosage form; (4) washing the oral dosage form with at least one flavored odor masking coating substance to improve user acceptance; and (5) drying the oral dosage form. A substance to improve user acceptance (i.e., flavorant, fragrance, and/or coloring agent), if not already in solution, may be combined into a solution with a volatile or other suitable solvent (e.g., isopropyl alcohol) and applied to at least a portion of the surface of an oral dosage form by a washing action. Use of a volatile solvent may allow more rapid drying. In an alternative embodiment of the present invention, a substance to improve user acceptance may be applied to an outer surface or inner surface, or both surfaces, of the oral dosage form.

In yet another presently preferred embodiment of the present invention, a method for improving user acceptance of an oral dosage form may include the steps of: (1) selecting an active ingredient for delivery to a user; (2) selecting an oral dosage form; (3) incorporating the active ingredient into the oral dosage form; (4) applying a flavored odor masking coating comprising at least one substance to improve user acceptance to an outer surface of the oral dosage form; and (5) drying the oral dosage form. A coating may be applied by use of a pan and/or spray (e.g., aerosol) mechanism. Moreover, multiple flavored odor masking coatings may be applied to an oral dosage form depending on the olfactory strength of the undesirable property needing to be neutralized and/or masked. In addition, a coating may be applied to at least a portion of the outer surface or the inner surface, or both surfaces, of an oral dosage form, if desired.

As further contemplated herein, one presently preferred embodiment of the present invention for improving user consumption of an oral dosage form containing an active ingredient for use as a supplement and/or medicament in a human may include the steps of: (1) selecting a capsule dosage form; (2) incorporating a quantity of marine fish oils containing high quantities of PUFA's into the capsule dosage form; and (3) applying at least one flavored odor masking coatings to at least a portion of the dosage form outer surface of the capsule to improve odor and improve human acceptance of the capsule.

One presently preferred embodiment of a dosage delivery apparatus of the present invention may include: (1) an oral dosage form having a receiving surface; (2) an active ingredient introduced into the oral dosage form; and (3) an odor masking substance applied to at least a portion of the receiving surface of the oral dosage form. The oral dosage form may be selected from the group consisting of a solid, liquid, powder, tablet, capsule, gel, soft gel, soft elastic capsule, granule, microgranule, emulsion, suspension, troche, tablet triturate, lozenge, and pill. Preferably, the oral dosage form comprises a soft gelatin capsule. The receiving surface of the oral dosage form may include the outer surface, the inner surface, or a combination of both surfaces of the oral dosage form to which the odor masking substance may be applied.

In one presently preferred embodiment of the present invention, the active ingredient is selected from the group consisting of nutritional supplement, natural product, pharmaceutical, food supplement, diagnostic agent, vitamin, mineral, botanical, herbal, and marine product. In addition, the active ingredient may have an unpleasant taste and/or noxious odor (i.e., fish oil). Preferably, the odor masking substance is selected from the group consisting of fragrance, flavorant, and coloring agent sufficient to mask the unpleasant taste and/or noxious odor of the active ingredient.

In one presently preferred embodiment the odor masking substance may comprise a water soluble or an oil soluble flavoring that may include, by example and not by way of limitation, a berry, strawberry, lemon, chocolate, vanilla, almond, blueberry, coconut, banana, orange, grape, cherry, lime, mint, or cinnamon flavor. Moreover, the odor masking substance may be combined with a solvent or excipient (e.g., isopropyl alcohol) to improve application to at least a portion of the receiving surface of the oral dosage form although not required. Accordingly, it will be appreciated that the present invention contemplates a methodology and practice which is directed to the addition of a substance or flavor to the oral dosage form (i.e., soft gel capsule) to improve the organoleptic qualities of the dosage form with or without excipients.

Past efforts by those skilled in the art have attempted to improve user acceptance of certain active ingredients with unpleasant odors or tastes by providing them in oral dosage forms with altered chemical compositions or altering the temperature of the dosage form. These past efforts have not been successful and the need to improve user acceptance of certain active ingredients with unpleasant flavors, odors, tastes, smells, and/or palatability still exists.

The novel dosage delivery apparatus and methods of the present invention for improving user acceptance by applying one or more flavored odor masking coatings to at least a portion of the surface (i.e., inner or outer) oral dosage form have not heretofore been identified or evaluated. The novel dosage delivery apparatus of the present invention does not require the incorporation of a substance into the oral dosage apparatus itself and therefore are not expected to alter the dissolution of the dosage form, nor impair the absorption of the active ingredient into the circulation of a user. Moreover, the novel dosage delivery apparatus of the present invention provide enhanced acceptance and consumption by a user, while remaining economical to manufacture and are therefore a significant advancement in the art.

As appreciated, the present invention may be embodied in other specific forms without departing from its spirit or essential characteristics. The described embodiments are to be considered in all respects only as illustrative, and not restrictive. The scope of the invention is, therefore, indicated by the appended claims, rather than by the foregoing description. All changes which come within the meaning and range of equivalency of the claims are to be embraced within their scope.

What is claimed and desired to be secured by United States Letters Patent is:

1. A method for improving user acceptance of an oral dosage form, the method comprising the steps of:
selecting an oral dosage form having a receiving surface;
introducing an active ingredient into the oral dosage form; and
applying an odor masking substance to at least a portion of the receiving surface of the oral dosage form.

2. A method as defined in claim 1, wherein the oral dosage form is selected from the group consisting of solid, liquid, powder, tablet, capsule, gel, soft gel, soft elastic capsule, granule, microgranule, emulsion, suspension, troche, tablet triturate, lozenge, and pill.

3. A method as defined in claim 1, wherein the active ingredient is selected from the group consisting of nutritional supplement, natural product, pharmaceutical, food supplement, diagnostic agent, vitamin, mineral, botanical, herbal, and marine product.

4. A method as defined in claim 1, wherein the active ingredient is a fish oil.

5. A method as defined in claim 1, wherein the active ingredient comprises an unpleasant taste.

6. A method as defined in claim 1, wherein the active ingredient comprises a noxious odor.

7. A method as defined in claim 1, wherein the odor masking coating is selected from the group consisting of fragrance, flavorant, and coloring agent.

8. A method as defined in claim 1, further comprising the step of combining the odor masking coating with a solvent to improve application to at least a portion of the receiving surface of the oral dosage form.

9. A method as defined in claim 1, wherein the odor masking substance comprises a water soluble flavoring.

10. A method as defined in claim 9, wherein the water soluble flavoring is selected from the group consisting of berry, strawberry, lemon, chocolate, vanilla, almond, blueberry, coconut, banana, orange, grape, cherry, lime, mint, and cinnamon.

11. A method as defined in claim 1, wherein the step of applying the odor masking substance is selected from the group consisting of polishing, washing, and spraying.

12. A method as defined in claim 11, wherein the step of polishing comprises the steps of polishing the oral dosage form in a tumbler apparatus, wherein one or more polishing cloths are saturated with at least one odor masking substance to apply the substance to at least a portion of the receiving surface of the oral dosage form.

13. A method as defined in claim 11, wherein the step of washing comprises the steps of washing the oral dosage form with at least one odor masking substance and drying the treated oral dosage form.

14. A method as defined in claim 1, further comprising the step of drying the treated oral dosage form.

15. A dosage delivery apparatus comprising:
an oral dosage form having a receiving surface;
an active ingredient introduced into the oral dosage form; and
an odor masking substance applied to at least a portion of the receiving surface of the oral dosage form.

16. A dosage delivery apparatus as defined in claim 15, wherein the active ingredient is selected from the group consisting of nutritional supplement, natural product, pharmaceutical, food supplement, diagnostic agent, vitamin, mineral, botanical, herbal, and marine product.

17. A dosage delivery apparatus as defined in claim 15, wherein the active ingredient is a fish oil.

18. A dosage delivery apparatus as defined in claim 15, wherein the active ingredient comprises an unpleasant taste.

19. A dosage delivery apparatus as defined in claim 15, wherein the active ingredient comprises a noxious odor.

20. A dosage delivery apparatus as defined in claim 15, wherein the odor masking substance is selected from the group consisting of fragrance, flavorant, and coloring agent.
